(19) United States
(12) Patent Application Publication
(10) Pub. No.: US 2026/0325820 A1
(43) Pub. Date: Oct. 22, 2026

(54) FLEXIBLE ROTOR BLADE FOR A RIGHT SIDED HEART PUMP

(71) Applicant: ABIOMED, INC., DANVERS, MA (US)

(72) Inventors: JAMES MELTON, DANVERS, MA (US); JAIRO LEANDRO MOUX, DANVERS, MA (US); CHARLES PERKINS, DANVERS, MA (US); NANDISH DESAI, DANVERS, MA (US)

(73) Assignee: ABIOMED, INC., DANVERS, MA (US)

(21) Appl. No.: 19/649,381

(22) Filed: Apr. 16, 2026

Related U.S. Application Data
(60) Provisional application No. 63/790,824, filed on Apr. 18, 2025.

(51) Int. Cl. A61M 60/165; A61M 60/13; A61M 60/216; A61M 60/808; A61M 60/829

(52) U.S. Cl. CPC A61M 60/165; A61M 60/13; A61M 60/216; A61M 60/808; A61M 60/829; A61M 2205/0216; A61M 2205/0238; A61M 2205/103; A61M 2205/3334; A61M 2205/50

(57) ABSTRACT

A system for insertion into a patient's vasculature includes a catheter, a pump assembly and a cannula. The pump assembly includes an inlet, an outlet, and a rotor disposed between the inlet and the outlet. At least one rotor blade is made of a flexible material and is fixedly connected to the rotor. The pump assembly is operable to cause fluid to flow through the pump assembly inlet, by or about the rotor and the at least one rotor blade, and through the pump assembly outlet. Should a biomaterial enter the pump assembly inlet with the fluid flow, the at least one rotor blade is sufficiently flexible to permit the biomaterial to deflect the at least one rotor blade to permit the fluid and biomaterial to flow by or about the rotor blade and through the pump assembly outlet.

### CROSS REFERENCE TO RELATED APPLICATIONS

This application claims the benefit of priority under 35 U.S.C. §119 to prior filed U.S. Provisional Patent Application No. 63/790,824, filed Apr. 18, 2025 (Attorney Docket No.: 266489.000003 (ABX0376USPSP1), the entire contents of which is hereby incorporated by reference in its entirety as if set forth in full herein.

### FIELD

The present disclosure relates generally to medical devices, and in particular to system for insertion into a patient's vasculature. More specifically, this disclosure relates to a blood pump, in particular an intravascular blood pump for percutaneous insertion into a patient's blood vessel, to support a blood flow in a patient's blood vessel. This disclosure particularly relates to a right ventricular blood pump to support a blood flow from the vena cava through the right ventricle into the pulmonary artery.

### BACKGROUND

Intravascular blood pumps are inserted into a patient's vessel such as the aorta or vena cava and through a cardiac valve by means of a catheter and can also be referred to as catheter pumps. A blood pump typically comprises a pump section with a blood flow inlet and a blood flow outlet. To cause blood flow from the blood flow inlet to the blood flow outlet, typically an impeller or rotor is rotatably supported within the pump casing about an axis of rotation for conveying blood. The blood pump may be driven by a motor included in the blood pump adjacent to the pump section or may alternatively be driven by a motor outside the patient's body, in which case the motor is connected to the impeller by a flexible drive shaft extending through the catheter.

A right ventricular blood pump is inserted through the inferior or superior vena cava through the right ventricle of a patient's heart into the pulmonary artery by means of a catheter. Typically, the blood flow inlet of the blood pump is placed inside the right atrium, vena cava or right ventricle, while the pump section extends through the tricuspid valve, the right ventricle and the pulmonary valve into the pulmonary artery.

Any blood clots or thrombi that occur may be conveyed from the vena cava to the pulmonary artery, which, however, does not cause severe harm to the patient because the thrombi only end up in the pulmonary circulation. More importantly, blood clots may clog the blood pump and thus may cause failure of the blood pump, which has to be avoided. Filters are known that can be placed in the vena cava, such as balls of random Nitinol wires. However, handling of such filters adjunctively to the blood pump therapy may be cumbersome, e.g., their insertion and particularly removal would require an additional access point. Any access point carries a risk of bleeding and infection. A correct alignment between blood pump and filter is necessary for performance and needs to be confirmed by means of visualization by echo or x-ray.

### SUMMARY

The disclosed technology includes a pump assembly that is operable to permit a biomaterial to enter through an inlet port(s) of the pump assembly, deflect a rotor blade of the pump assembly, and exit through an outlet port(s) of the pump assembly.

There is provided, in accordance with the disclosed technology, a system for insertion into a patient's vasculature. The system includes a catheter, a pump assembly and a cannula. The catheter has a proximal end, and a distal end, and extends along a longitudinal axis. The pump assembly is disposed at the distal end of the catheter. The pump assembly includes an inlet, an outlet, and a rotor disposed between the inlet and the outlet. The pump assembly includes at least one rotor blade that is made of a flexible material and is fixedly connected to the rotor. The at least one rotor blade is moveable between a crimped state and a running state. The cannula is connected to the distal end portion of the pump assembly. The cannula has a proximal cannula portion that includes one or more cannula inlet ports, and a distal cannula portion that includes one or more cannula outlet ports. In the running state, the pump assembly is operable to cause fluid to flow through the pump assembly inlet, by or about the rotor and the at least one rotor blade, and through the pump assembly outlet. Should a biomaterial enter the pump assembly inlet with the fluid flow, the at least one rotor blade is sufficiently flexible to permit the biomaterial to flow through the pump assembly inlet, by or about the at least one rotor blade thereby deflecting the at least one rotor blade to permit the fluid and biomaterial to flow by or about the rotor blade and through the pump assembly outlet.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

FIG. 1 is a schematic pictorial illustration of a catheter inserted into a patient's heart, in accordance with the disclosed technology;

FIG. 2 is a schematic pictorial illustration of a medical system including the catheter of FIG. 1 including a controller in accordance with the disclosed technology;

FIG. 3A is a schematic pictorial illustration of a detail view of Detail FIG. 3 in FIG. 2, showing a biomaterial entering a pump inlet in accordance with the disclosed technology;

FIG. 3B is a schematic pictorial illustration of a detail view of Detail FIG. 3 in FIG. 2, showing a biomaterial passing by a pump rotor blade in accordance with the disclosed technology;

FIG. 3C is a schematic pictorial illustration of a detail view of Detail FIG. 3 in FIG. 2, showing a biomaterial passing by a pump rotor blade in accordance with the disclosed technology;

FIG. 3D is a schematic pictorial illustration of a detail view of Detail FIG. 3 in FIG. 2, showing a biomaterial exiting a pump outlet in accordance with the disclosed technology;

FIG. 4 is a plan view of a pump rotor and rotor blade in accordance with the disclosed technology;

FIG. 5 is a flow chart of a method of using a catheter, according to aspects of the present disclosure.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g. “about 90%” may refer to the range of values from 81% to 99%.

In addition, as used herein, the terms “patient,” “host,” “user,” and “subject” refer to any human or animal subject and are not intended to limit the systems or methods to human use, although use of the subject invention in a human patient represents a preferred embodiment. As well, the term “proximal” indicates a location closer to the operator whereas “distal” indicates a location further away to the operator or physician.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein. Such modifications and variations are intended to be included within the scope of the claims.

Alternative apparatus and system features and alternative method steps are presented in example embodiments herein. Each given example embodiment presented herein can be modified to include a feature and/or method step presented with a different example embodiment herein where such feature and/or step is compatible with the given example as understood by a person skilled in the pertinent art as well as where explicitly stated herein.

The disclosed technology relates to a catheter (e.g., an intravascular pump) that is configured to cause a preferential flow of blood and any biomaterial flowing with the blood flow. To help the reader better understand the disclosed technology, the catheter will be described, in general, first and then various devices/methods for creating the preferential flow will be described in relation to the intravascular pump. It will be appreciated, however, that the disclosed technology can be applicable to other intravascular pumps having differing designs from that described herein. Furthermore, the disclosed technology can be applicable to other catheters in which entry of biomaterial into a pump is common. Accordingly, the disclosed technology should not be limited to the specific catheter shown in the drawings and described herein.

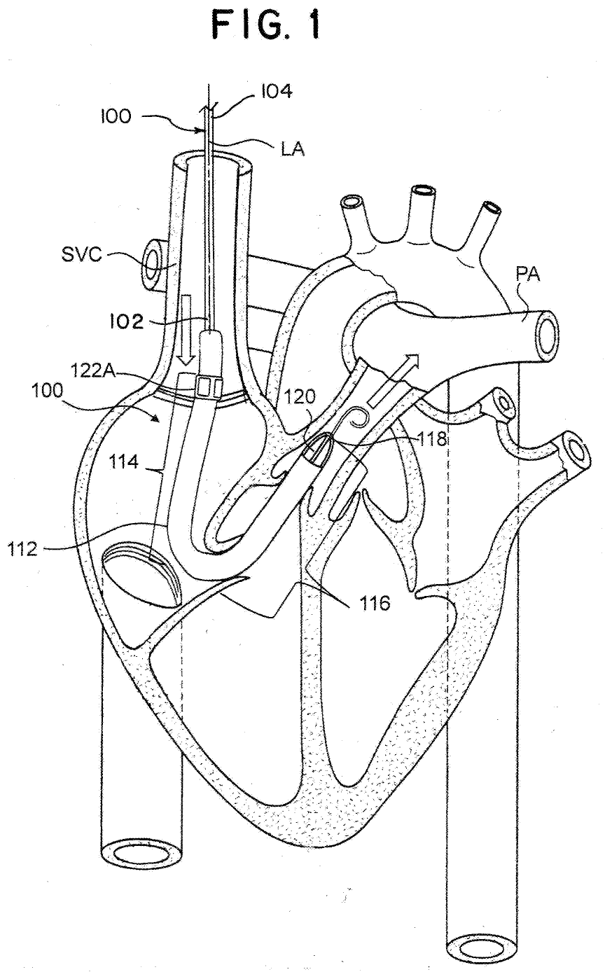

Referring now to FIG. 1, a system for insertion into a patient's vasculature is illustrated. As illustrated in FIG. 1, the system is configured to be positioned such that the pump inlet is disposed in a superior vena cava (“SVC”) and the blood flow outlet is disposed in a pulmonary artery (“PA”). The system includes a catheter 100 that has a proximal end 104, and a distal end 102. Catheter 100 extends along a longitudinal axis (“LA”). The system includes a pump assembly 108 that is disposed at the distal end 102 of the catheter 100. Pump assembly 108 includes an inlet, an outlet, and a rotor 109A disposed between the inlet and the outlet. The pump assembly includes at least one rotor blade 109B. Rotor blades 109B is made of a flexible material and is fixedly connected to the rotor. As discussed in more detail below, the at least one rotor blade 109B is moveable between a bypass state and a running state.

A cannula 112 is connected to the distal end portion of the pump assembly 108. Cannula 112 has a proximal cannula portion 114 and a distal cannula portion 116. Proximal cannula portion 114 has one or more cannula inlet ports 122. Distal cannula portion 116 has one or more cannula outlet ports 120. In the running state, the pump assembly is operable to cause fluid to flow through the pump assembly inlet, by or about the rotor and the at least one rotor blade, and through the pump assembly outlet. The at least one rotor blade 109B is sufficiently flexible to permit a biomaterial (“THR”) flowing with the fluid to flow through the pump assembly inlet, by or about the at least one rotor blade thereby deflecting the at least one rotor blade to permit the fluid and biomaterial to flow by or about the rotor blade and through the pump assembly outlet. In one example, the at least one rotor blade 109B can have a hydrophobic coating on the exterior surface of the rotor blade.

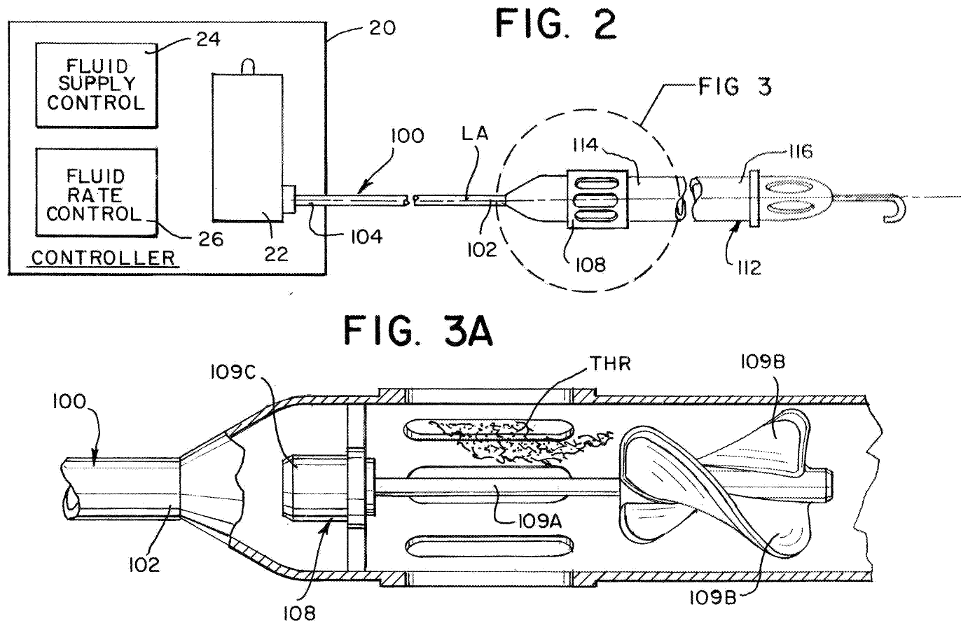

As shown in FIG. 2, a controller 20 is connected to the proximal end of the catheter body and configured to operate the pump assembly. Controller 20 comprises a fluid reservoir 22 and a fluid supply control 24 and a fluid rate control 26. Fluid reservoir 22 is configured to contain a hemocompatible fluid. Fluid supply control 24 is configured to control the supply of the hemocompatible fluid to the pump assembly. Fluid rate control 26 is configured to controlling operation of the pump assembly. Initially, as is known in the art, a purge cassette can be included with controller 20 to connect to a purge solution bag (not shown). A biocompatible purge solution is pumped from the bag, through the catheter and then through the bearings in the motor of the pump to prevent buildup in the bearings as well as to cool the motor. After the initial purge or flushing, controller 20 is configured to operate the pump assembly to pull a fluid, such as, for example, blood, through the pump inlet, by or about the at least one rotor blade 109B and through the pump outlet.

A pump assembly 108 is configured to be inserted into a patient's vasculature. The pump assembly can be advanced into the right heart from a puncture site in the femoral artery of a patient at a puncture site. In some other examples, the pump can be advanced from the internal jugular vein or the subclavian artery. As shown in FIG. 2, pump assembly 108 includes an inlet, and an outlet that is disposed opposite the inlet along longitudinal axis LA. A rotor 109A is disposed between the inlet and the outlet. At least one rotor blade 109B is fixedly connected to rotor 109A. The pump section 108 further includes a motor 109C. Motor 109C is configured to drive the rotor 109A. Alternatively, the motor 109C may located outside the patient's body, in which case the motor is connected to rotor 109A by a flexible drive shaft (not shown) extending through the catheter. Motor 109C is connected to rotor 109A and is operably connected to controller 20 to control the rate of rotation of rotor 109A, and thus at least one rotor blade 109B to pump blood from the inlet to the outlet.

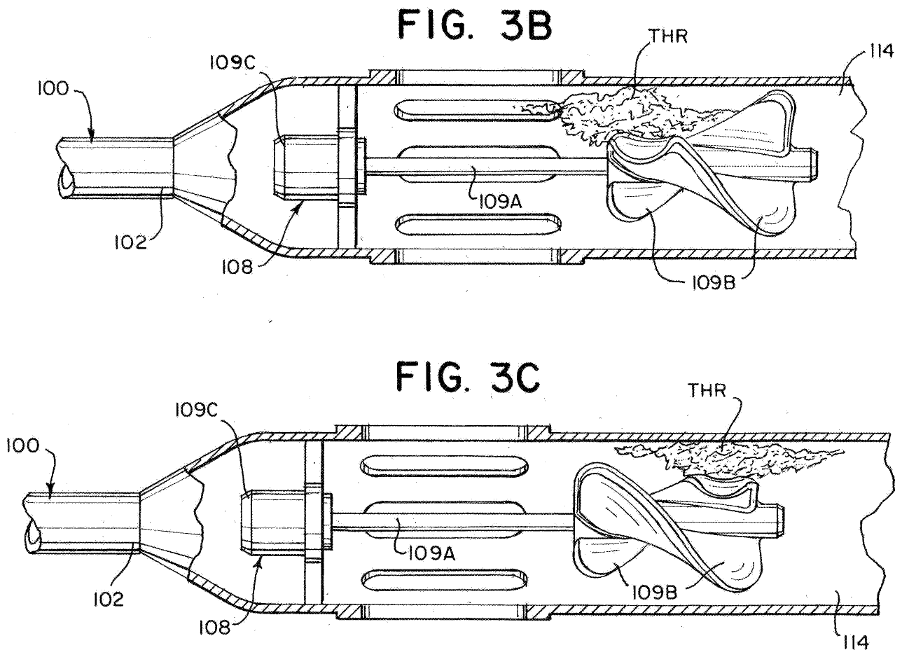

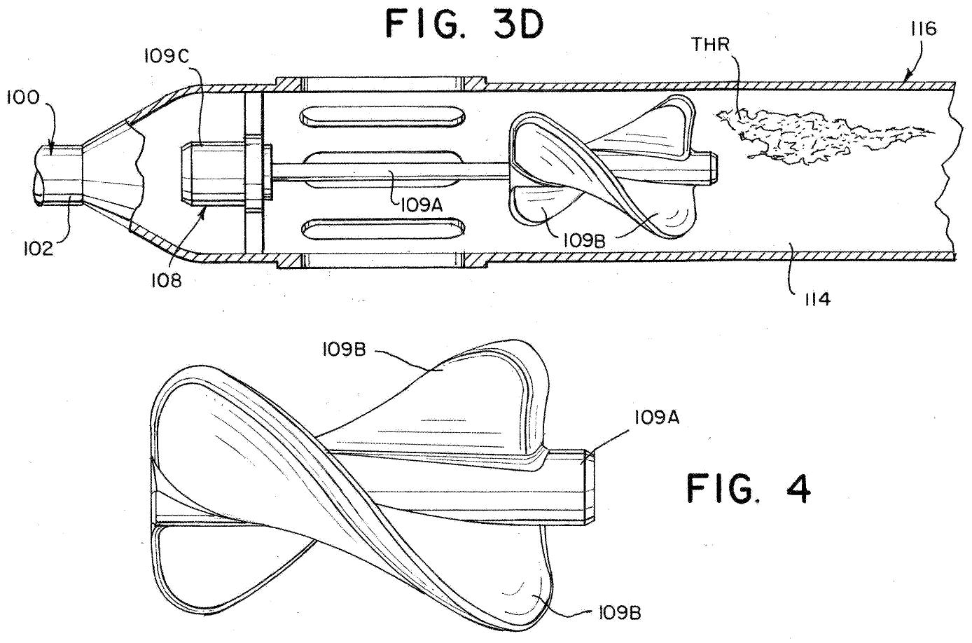

As shown in FIGS. 3A-3D, and FIG. 4, the rotor blade, in one example, can include two rotor blades 109B. Rotor blade 109B is made of a flexible material and is moveable between a running state and a bypass state. In the running state, pump assembly 108 causes fluid to flow through the pump assembly inlet, by or about the rotor 109A and the at least one rotor blade 109B, and through the pump assembly outlet. In the bypass state, the at least one rotor blade 109B is deformable to permit a biomaterial (THR), such as, for example, a thrombus, flowing with the fluid to flow through the pump assembly inlet, by or about the at least one rotor blade and through the pump assembly outlet. It is to be noted that in the bypass state, pump assembly 108 still causes fluid to flow through the pump assembly inlet, by or about the rotor 109A and the at least one rotor blade 109B, and through the pump assembly outlet along with the biomaterial THR. As shown in FIGS. 3B and 3C, if needed, the biomaterial THR can deform the shape of rotor blade 109B thereby permitting the biomaterial to pass by the rotor rather than getting stuck at the rotor and thereby risking pump failure. For example, the biomaterial THR could wrap around the rotor and/or the rotor blade, or become wedged between rotor, rotor blade and the inner wall of the pump housing thereby preventing the rotor and rotor blade from rotating and causing pump failure. The rotor blade 109B can have different shapes and the flexibility of the rotor blade 109B can vary. In one example, the rotor blade 109B can have different flexibilities as the blade transitions from closer to the axis of rotation radially outwardly to the radial outermost portion of the rotor blade. In some examples, the rotor blade 109B can have a tangential blade shape such that the blades that are oriented parallel to the shaft, creating a tangential flow of fluid as the fluid moves through the impeller. Tangential blades are designed to move the fluid along the circumference of the impeller, rather than radially outwards or axially. Thus, a tangential blade can be used to increase positive fluid displacement while still permitting a biomaterial to pass by, through or about the impeller blade.

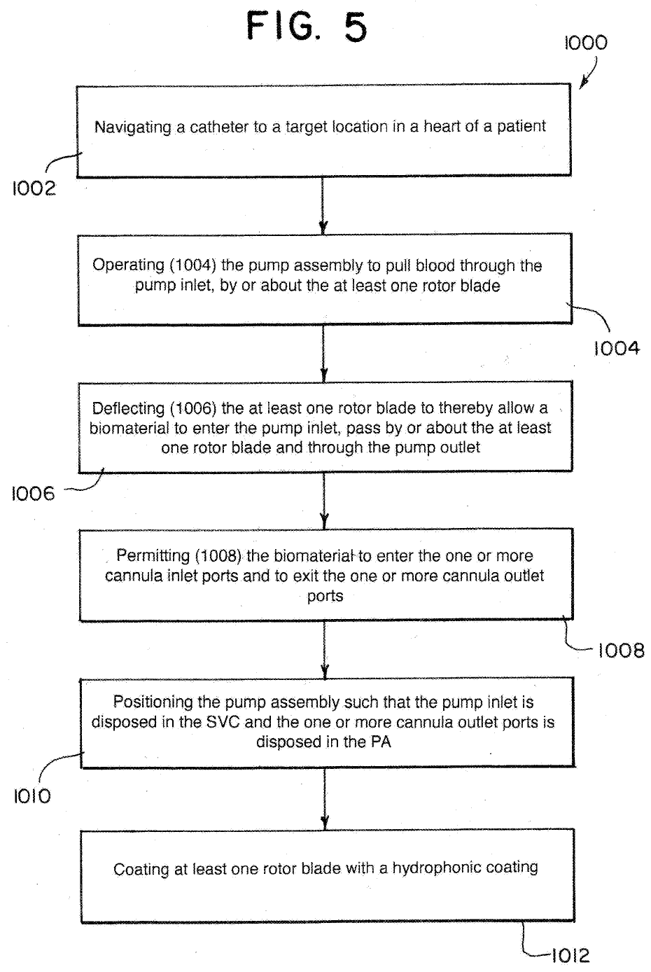

Referring now to FIG. 5, a method 1000 of performing a medical procedure comprises the following steps. At step 1002, a catheter is navigated to a target location in a heart of a patient. The catheter extends along a longitudinal axis and comprises a catheter body. The catheter comprises a distal end and a proximal end. A pump assembly is disposed at the distal end of the catheter body. The pump assembly includes a pump inlet and a pump outlet. A cannula 112 is connected to the distal end portion of the pump assembly. The cannula comprises a proximal cannula portion 114 comprising one or more cannula inlet ports 122, and a distal cannula portion 116 comprising one or more cannula outlet ports 120.

At step 1004, the pump assembly is operated to pull blood through the pump inlet, by or about the at least one rotor blade, and through the pump outlet.

At step 1006, the at least one rotor blade is deflected to thereby allow a biomaterial to enter the pump inlet, pass by or about the at least one rotor blade and through the pump outlet.

At step 1008, the biomaterial is permitted to enter the one or more cannula inlet ports and to exit the one or more cannula outlet ports.

In one example, the biomaterial is a thrombus. The system is configured to be positioned such that the pump inlet is disposed in a superior vena cava SVC and the one or more cannula outlet ports 120 are disposed in a pulmonary artery PA.

When navigating the catheter to the target location, the catheter is navigated through the SVC, through the right atrium RA, and through the pulmonary valve PV into the pulmonary artery PA.

In accordance with an additional step, the pump assembly is positioned such that the pump inlet is disposed in the SVC and the one or more cannula outlet ports is disposed in the PA.

In accordance with an additional step, the at least one rotor blade is coated with a hydrophobic coating.

The following clauses list non-limiting examples in accordance with the disclosed technology:
Clause 1: A system for insertion into a patient's vasculature, the system comprising:
   a catheter (100) comprising a proximal end (104), and a distal end (102), the catheter extending along a longitudinal axis (LA) and;
   a pump assembly (108) disposed at the distal end of the catheter and comprising an inlet, an outlet, and a rotor (109A) disposed between the inlet and the outlet, the pump assembly including at least one rotor blade (109B) being made of a flexible material and connected to the rotor, the at least one rotor blade being moveable between a crimped state and a running state; and
   a cannula (112) connected to the distal end portion of the pump assembly, the cannula comprising a proximal cannula portion (114) comprising one or more cannula inlet ports (122); and a distal cannula portion (116) comprising one or more cannula outlet ports (120);
   wherein in the running state, the pump assembly being operable to cause fluid to flow through the pump assembly inlet, by or about the rotor and the at least one rotor blade, and through the pump assembly outlet; the at least one rotor blade being sufficiently flexible to permit a biomaterial (THR) flowing with the fluid to flow through the pump assembly inlet, by or about the at least one rotor blade thereby deflecting the at least one rotor blade to permit the fluid and biomaterial to flow by or about the rotor blade and through the pump assembly outlet.
Clause 2: The system according to clause 1, wherein the system is configured to be positioned such that the pump inlet is disposed in a superior vena cava (SVC) and the blood flow outlet (120) is disposed in a pulmonary artery (PA).
Clause 3: The system according to any of clauses 1-2, further comprising a controller (20) connected to the proximal end of the catheter and configured to operate the pump assembly.
Clause 4: The system according to any of clauses 1-3, wherein the controller comprises a fluid reservoir (22) that is configured to contain a hemocompatible fluid, the controller being configured to deliver the hemocompatible fluid through the pump assembly.
Clause 5: The system according to any of clauses 1-4, wherein the controller comprises a fluid supply control (24) for controlling supply of the hemocompatible fluid to the pump assembly and a flow rate control (26) for controlling operation of the pump assembly.
Clause 6: The system according to any of clauses 1-4, wherein the controller is configured to operate the pump assembly to pull a fluid through the pump inlet, by or about the at least one rotor blade and through the pump outlet.
Clause 7: The system according to any of clauses 1-6, wherein the at least one rotor blade comprises a hydrophobic coating.
Clause 8: A method (1000) of performing a medical procedure comprising:
   navigating (1002) a catheter to a target location in a heart of a patient, the catheter extending along a longitudinal axis and comprising a catheter body comprising a distal end and a proximal end; a pump assembly disposed at the distal end of the catheter body, the pump assembly including a pump inlet and a pump outlet, and a cannula (112) connected to the distal end portion of the pump assembly, the cannula comprising a proximal cannula portion (114) comprising one or more cannula inlet ports (122), and a distal cannula portion (116) comprising one or more cannula outlet ports (120);
   operating (1004) the pump assembly to pull blood through the pump inlet, by or about the at least one rotor blade, and through the pump outlet;
   deflecting (1006) the at least one rotor blade to thereby allow a biomaterial to enter the pump inlet, pass by or about the at least one rotor blade and through the pump outlet; and
   permitting (1008) the biomaterial to enter the one or more cannula inlet ports and to exit the one or more cannula outlet ports.
Clause 9: The method according to clause 8, wherein the biomaterial is a thrombus.
Clause 10: The method according to clauses 8 or 9, wherein the system is configured to be positioned such that the pump inlet is disposed in a superior vena cava (SVC) and the one or more cannula outlet ports (120) being disposed in a pulmonary artery (PA).
Clause 11: The method according to any of clauses 8-10, wherein navigating the catheter to the target location comprises navigating the catheter through the superior vena cava (SVC), through the right atrium (RA), and through the pulmonary valve (PV) into the pulmonary artery (PA).
Clause 12: The method according to any of clauses 8-11, further comprising the step of: positioning the pump assembly such that the pump inlet is disposed in the SVC and the one or more cannula outlet ports is disposed in the PA.
Clause 13: The method according to any of clauses 8-12, further comprising the step of coating at least one rotor blade with a hydrophobic coating.
Clause 14: A pump assembly (108) configured to be inserted into a patient's vasculature comprising:
   an inlet;
   an outlet, opposite the inlet along a longitudinal axis;
   a rotor (109A) disposed between the inlet and the outlet, comprising:
      at least one rotor blade (109B) being made of a flexible material and connected to the rotor, the at least one rotor blade being moveable between a running state and a bypass state;
      in the running state, the pump assembly causing fluid to flow through the pump assembly inlet, by or about the rotor and the at least one rotor blade, and through the pump assembly outlet;
      in the bypass state the at least one rotor blade deforming to permit a biomaterial (THR) flowing with the fluid to flow through the pump assembly inlet, by or about the at least one rotor blade and biomaterial to flow by or about the rotor blade and through the pump assembly outlet.
Clause 15: The pump assembly according to clause 14, wherein the pump assembly is configured to be positioned such that the pump inlet is disposed in a superior vena cava (SVC) and the blood flow outlet (114) flows into a pulmonary artery (PA).
Clause 16: The pump assembly according to any of clauses 14-15, further comprising a controller (20) connected to the pump assembly inlet and configured to operate the pump assembly.
Clause 17: The pump assembly according to any of clauses 14-16, wherein the controller comprises a fluid reservoir (22) that is configured to contain a hemocompatible fluid, the controller being configured to deliver the hemocompatible fluid through the pump assembly.
Clause 18: The pump assembly according to any of clauses 14-17, wherein the controller comprises a fluid supply control (24) for controlling supply of the hemocompatible fluid to the pump assembly and a flow rate control (26) for controlling operation of the pump assembly.
Clause 19: The pump assembly according to any of clauses 14-18, wherein the controller is configured to operate the pump assembly to pull a fluid through the pump assembly inlet, by or about the at least one rotor blade and through the pump assembly outlet.
Clause 20: The pump assembly according to any of clauses 14-19, wherein the at least one rotor blade comprises a hydrophobic coating.

The descriptions contained herein are examples of embodiments of the invention and are not intended in any way to limit the scope of the invention. Modifications and variations apparent to those having skilled in the pertinent art according to the teachings of this disclosure are intended to be within the scope of the claims which follow.

What is claimed is:

1. A system for insertion into a patient's vasculature, the system comprising:
a catheter comprising a proximal end, and a distal end, the catheter extending along a longitudinal axis and;
a pump assembly disposed at the distal end of the catheter and comprising an inlet, an outlet, and a rotor disposed between the inlet and the outlet, the pump assembly including at least one rotor blade being made of a flexible material and connected to the rotor, the at least one rotor blade being moveable between a crimped state and a running state; and
a cannula connected to the distal end portion of the pump assembly, the cannula comprising a proximal cannula portion comprising one or more cannula inlet ports; and a distal cannula portion comprising one or more cannula outlet ports;
wherein in the running state, the pump assembly being operable to cause fluid to flow through the pump assembly inlet, by or about the rotor and the at least one rotor blade, and through the pump assembly outlet; the at least one rotor blade being sufficiently flexible to permit a biomaterial flowing with the fluid to flow through the pump assembly inlet, by or about the at least one rotor blade thereby deflecting the at least one rotor blade to permit the fluid and biomaterial to flow by or about the rotor blade and through the pump assembly outlet.

2. The system according to claim 1, wherein the system is configured to be positioned such that the pump inlet is disposed in a superior vena cava and the blood flow outlet is disposed in a pulmonary artery.

3. The system according to claim 1, further comprising a controller connected to the proximal end of the catheter and configured to operate the pump assembly.

4. The system according to claim 3, wherein the controller comprises a fluid reservoir that is configured to contain a hemocompatible fluid, the controller being configured to deliver the hemocompatible fluid through the pump assembly.

5. The system according to claim 4, wherein the controller comprises a fluid supply control for controlling supply of the hemocompatible fluid to the pump assembly and a flow rate control for controlling operation of the pump assembly.

6. The system according to claim 4, wherein the controller is configured to operate the pump assembly to pull a fluid through the pump inlet, by or about the at least one rotor blade and through the pump outlet.

7. The system according to claim 1, wherein the at least one rotor blade comprises a hydrophobic coating.

8. A method of performing a medical procedure comprising:
navigating a catheter to a target location in a heart of a patient, the catheter extending along a longitudinal axis and comprising a catheter body comprising a distal end and a proximal end; a pump assembly disposed at the distal end of the catheter body, the pump assembly including a pump inlet and a pump outlet, and a cannula connected to the distal end portion of the pump assembly, the cannula comprising a proximal cannula portion comprising one or more cannula inlet ports, and a distal cannula portion comprising one or more cannula outlet ports;
operating the pump assembly to pull blood through the pump inlet, by or about the at least one rotor blade, and through the pump outlet;
deflecting the at least one rotor blade to thereby allow a biomaterial to enter the pump inlet, pass by or about the at least one rotor blade and through the pump outlet; and
permitting the biomaterial to enter the one or more cannula inlet ports and to exit the one or more cannula outlet ports.

9. The method according to claim 8, wherein the biomaterial is a thrombus.

10. The method according to claim 8, wherein the system is configured to be positioned such that the pump inlet is disposed in a superior vena cava and the one or more cannula outlet ports being disposed in a pulmonary artery.

11. The method according to claim 10, wherein navigating the catheter to the target location comprises navigating the catheter through the superior vena cava, through the right atrium, and through the pulmonary valve into the pulmonary artery.

12. The method according to claim 11, further comprising the step of:
positioning the pump assembly such that the pump inlet is disposed in the SVC and the one or more cannula outlet ports is disposed in the PA.

13. The method according to claim 8, further comprising the step of coating at least one rotor blade with a hydrophobic coating.

14. A pump assembly configured to be inserted into a patient's vasculature comprising:
an inlet;
an outlet, opposite the inlet along a longitudinal axis;
a rotor disposed between the inlet and the outlet, comprising:
at least one rotor blade being made of a flexible material and connected to the rotor, the at least one rotor blade being moveable between a running state and a bypass state;
in the running state, the pump assembly causing fluid to flow through the pump assembly inlet, by or about the rotor and the at least one rotor blade, and through the pump assembly outlet;
in the bypass state the at least one rotor blade deforming to permit a biomaterial flowing with the fluid to flow through the pump assembly inlet, by or about the at least one rotor blade and biomaterial to flow by or about the rotor blade and through the pump assembly outlet.

15. The pump assembly according to claim 14, wherein the pump assembly is configured to be positioned such that the pump inlet is disposed in a superior vena cava and the blood flow outlet flows into a pulmonary artery.

16. The pump assembly according to claim 14, further comprising a controller connected to the pump assembly inlet and configured to operate the pump assembly.

17. The pump assembly according to claim 15, wherein the controller comprises a fluid reservoir that is configured to contain a hemocompatible fluid, the controller being configured to deliver the hemocompatible fluid through the pump assembly.

18. The pump assembly according to claim 17, wherein the controller comprises a fluid supply control for controlling supply of the hemocompatible fluid to the pump assembly and a flow rate control for controlling operation of the pump assembly.

19. The pump assembly according to claim 18, wherein the controller is configured to operate the pump assembly to pull a fluid through the pump assembly inlet, by or about the at least one rotor blade and through the pump assembly outlet.

20. The pump assembly according to claim 14, wherein the at least one rotor blade comprises a hydrophobic coating.
